(19) United States
(12) Patent Application Publication
(10) Pub. No.: US 2015/0272553 A1
(43) Pub. Date: Oct. 1, 2015

(54) ULTRASOUND PROBE FOR AUTOMATED MANAGEMENT OF DATA ENTRY FOR A PATIENT

(71) Applicant: General Electric Company, Schenectady, NY (US)

(72) Inventors: Jithin THATTARI KANDIYIL, Bangalore (IN); Nachiyappan LAKSHMANAN, Bangalore (IN)

(21) Appl. No.: 14/670,442

(22) Filed: Mar. 27, 2015

(30) Foreign Application Priority Data
Apr. 1, 2014 (IN) 1734/CHE/2014

(51) Int. Cl. A61B 8/00

(52) U.S. Cl. CPC A61B 8/585; A61B 8/54; A61B 8/565; A61B 8/461

(57) ABSTRACT

A system for automated management of data entry for a patient in an ultrasound device is disclosed. The system includes an ultrasound probe having a wireless reader capable of reading data including one or more of patient information and patient health history from an electronically readable card associated with the patient. Then a controller is present to configure the ultrasound probe for performing the ultrasound scanning based on set-up information and transmit the data to a remote location. A central server is present in the remote location for receiving the data from the controller and communicating the set-up information to the controller based on the data.

### FIELD OF THE INVENTION

The subject matter disclosed herein relates to management of data entry for patients. More specifically the subject matter relates to automated management of data entry for patients in an ultrasound device.

### BACKGROUND OF THE INVENTION

Hospital environment needs patients to be registered into the hospital with patient health history and patient personal information. Presently when a patient enters the hospital, patient's health history and other information need to be stored in the hospital network manually so that the patient can be admitted. The health history and patient information may be stored in a remote server and accessible to multiple hospitals in a locality or country. Thereafter if any scanning or tests needs to be performed on the patient then the patient health history and information need to be collected to perform these procedures which renders the whole process time consuming and cumbersome for the hospital. Moreover the situation may also affect the patient as they need to wait for a longer time to get the patient registration completed and then perform tests or scans. Multiple patients need to be attended at the same time frequently so the process of performing the scan or tests may get delayed and also render it to be very cumbersome for the hospital authorities to handle these cases efficiently.

Further medical devices such as big ultrasound devices may need to be moved from one bed to another bed to attend different patients and also to operation theatres which makes it cumbersome for the users.

Accordingly, a need exists for a system for providing improved accessibility of the medical device for the patient with portability in a hospital environment.

### SUMMARY OF THE INVENTION

The object of an embodiment of the present invention to provide a system providing improved accessibility of the medical device for the patient with portability in a hospital environment, which overcomes one or more drawbacks of the prior art. This is achieved by a system for automated management of data entry for patients for patient registration in a hospital environment as defined in the independent claim.

One advantage with the disclosed is that automated data entry for patients can be performed when the patient is bought for scanning (such as ultrasound scanning procedure) or performing the tests and devices used for performing the scan or tests are easily set up or configured based on the patient health history and information.

In an embodiment a system for automated management of data entry for a patient in an ultrasound device is disclosed. The system includes an ultrasound probe having a wireless reader capable of reading data including one or more of patient information and patient health history from an electronically readable card associated with the patient. Then a controller is present to configure the ultrasound probe for performing the ultrasound scanning based on set-up information and transmit the data to a remote location. A central server is present in the remote location for receiving the data from the controller and communicating the set-up information to the controller based on the data.

A more complete understanding of the present invention, as well as further features and advantages thereof, will be obtained by reference to the following detailed description and drawings.

### BRIEF DESCRIPTION OF THE DRAWINGS

FIG. 1 is a schematic illustration of a system for automated management of data entry for a patient in an ultrasound device in accordance with an embodiment;

FIG. 2 illustrates an ultrasound probe in accordance with an embodiment;

FIG. 3 is a schematic illustration of a perspective view of a patient table assembly holding a patient in accordance with an embodiment; and

FIG. 4 illustrates a method for managing automated patient data entry in an ultrasound device according to an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical and other changes may be made without departing from the scope of the embodiments. The following detailed description is, therefore, not to be taken as limiting the scope of the invention.

As discussed in detail below, embodiments of a system for automated management of data entry for a patient in an ultrasound device is disclosed. The system includes an ultrasound probe having a wireless reader capable of reading data including one or more of patient information and patient health history from an electronically readable card associated with the patient. Then a controller is present to configure the ultrasound probe for performing the ultrasound scanning based on set-up information and transmit the data to a remote location. A central server is present in the remote location for receiving the data from the controller and communicating the set-up information to the controller based on the data.

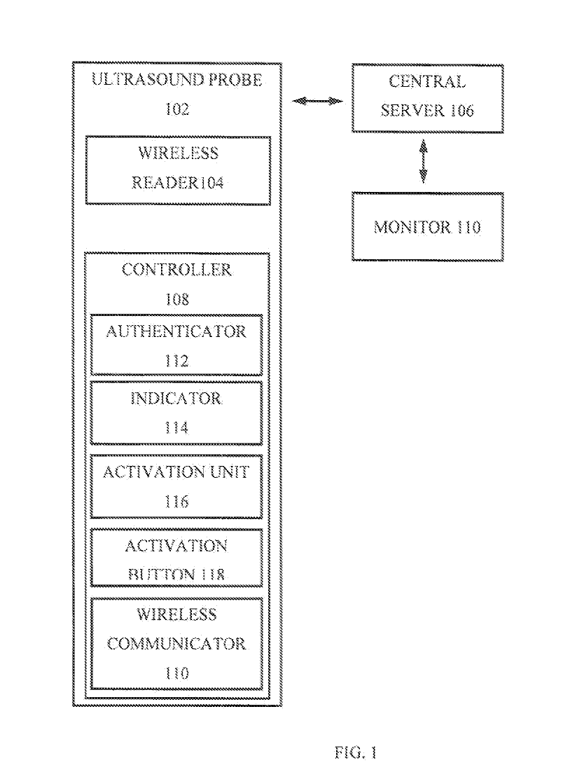

FIG. 1 is a system 100 for automated management of data entry for a patient in an ultrasound device in accordance with an embodiment. The data entry of the patient is performed to register patient health history and patient information in a database. The patient health history may include but are not limited to illness of the patient, different health criticalities, medications taken, tests and scans performed in the past, general health parameters for example blood pressure, sugar level and heart rate and so on. Further patient information may include but not limited to, name, nationality, origin, body weight, body structure, place, doctors consulted, institutes from where treatments undertaken in the past, age, sex, type of imaging (i.e. ultrasound, x-ray, PET scan, CT scan, MR scan and so on) or tests to be done based on the health condition of the patient and so on. The system 100 and its operations for automated management of data entry for a patient are described herein in the context of an ultrasound imaging environment according to an embodiment. However it may be envisioned that the system 100 may be used to operate in other forms of imaging or testing environments such as magnetic resonance (MR) imaging, x-ray imaging, positron emission tomography (PET) imaging, computed tomography (CT) imaging and so on. The system 100 includes an ultrasound probe 102 having a wireless reader 104 capable of reading patient data from an electronically readable card (not shown in FIG. 1) of the patient or a bed. The patient data may include but not limited to the patient health history and the patient information. The electronically readable card may be issued by a hospital that includes all records and data of the patient. The same electronically readable card may be tagged only to a particular patient or bed and can be used wherever the patient goes for treatment. The electronically readable card will have but not limited to an IP address of a monitor allocated for the bed, and the patient data. The wireless reader 104 may be a near field communication (NFC) reader, a RFID reader, a QR reader and a barcode reader. In case if this is a NFC reader then electronically readable card can be detected when the card is in NFC range of the ultrasound probe 102. Further the RFID reader can also read the electronically readable card when it is bought near to the ultrasound probe 102 within a RFID range. Now in case of the QR reader and the barcode reader the electronically readable card may include QR codes and barcodes respectively. The patient data may be linked to the QR codes or the barcode and stored in the electronically readable card. Alternatively only QR codes or the barcode of the patient may be stored in the electronically readable card and the patient data may be stored in a remote server and this is fetched are received based on the QR codes and the barcode. In an embodiment the electronically readable card may be swiped with the ultrasound probe 102 for reading the card.

Based on the patient data the ultrasound probe 102 set-up information may be received from a central server 106. The ultrasound probe 102 includes a controller 108 that communicates with the central server 106 to send the patient data to central sever 106. The communication between the controller 108 and the central server 106 may be through but not limited to a Wi-Fi network, a wireless LAN network, a 3G network and 4G network. The central sever 106 transmits the set-up information based on the patient data to the controller 108. The set-up information in the context of ultrasound imaging may include imaging presets, imaging parameters, health parameters to be monitored for the patient, number of scans to be performed, dosage and so on. The imaging presets may include for example imaging depth, intensity of imaging signals, type of imaging (such as cardiac, abdominal, obstetric etc.), and other imaging parameters based on the body structure of the patient. The set-up information received by the controller 108 are used to configure or set-up the ultrasound probe 102. Thus the same set-up information can be retrieved from the central sever 106 and used for setting-up an ultrasound probe for performing scanning in any location. The ultrasound probe 102 includes a wireless communicator 110 that facilitates communication between the ultrasound probe 102 and the central server 106. The wireless communicator 110 may function based on for example a Wi-Fi technique, a wireless LAN technique, a 3G communication technique, and a 4G communication technique. Now the ultrasound probe 102 is ready to be used on a patient for performing the desired ultrasound scanning tests.

A person (also referred to as “user”) using the ultrasound probe 102 such as a technician or a doctor may need to be authenticated. To this end the controller 108 includes an authenticator 112 that authenticates the person for using the ultrasound probe 102. In an embodiment the wireless reader 104 may be configured to read authentication data from an electronically readable card of the person. The authentication data is stored in the electronically readable card and this data is retrieved using a NFC communication technique, a QR or a barcode reading technique, and a RFID reading technique. The wireless reader 104 sends the authentication data to the authenticator 112 that reviews this data and authenticates the person. The authenticator 112 may use different techniques to authenticate the person. The authentication data may be compared with data associated with different persons stored in a look-up table form according to an exemplary embodiment. It may be envisioned that different ways and techniques for authenticating the person may be used.

The ultrasound probe 102 is used for capturing ultrasound images from the patient which is communicated to the central server 106. In an embodiment the ultrasound images may be processed in the ultrasound probe 102 and transmitted to the central server 106 by the wireless communicator 110 over a communication network. These ultrasound images may be used for future reference for the doctor.

Further the ultrasound probe 102 may also include an indicator 114 for indicating occurrence of some events to the user. The events may include the patient data being successfully read from the electronically readable card, sending of the patient data to the central server 106 and the patient data being received from the central server 106. The indicator 114 may be an audible sound such as beep sound. In an embodiment the indicator 114 may be a message provided on a display of the ultrasound probe 102. In another embodiment the indicator 114 may be an LED indicator. The indicator 114 enables the user to know about the status of the transmission of patient data within the ultrasound probe 102 conveniently.

The ultrasound probe 102 may also include an activation unit 116 configured to activate and deactivate the wireless reader 104 based on the user input. An activation button 118 may be utilized by the user for entering the user input. The activation button 118 may be provided at an accessible location of the ultrasound probe 102. An ultrasound probe such as an ultrasound probe 200 may also include one or more navigation keys such as a navigation key 202 and a touch input display 204 as shown in FIG. 2 according to an exemplary embodiment. The ultrasound probe 200 as shown in FIG. 2 is merely exemplary and therefore other ultrasound probes having different arrangement of the touch input display and navigation keys are within the scope of this disclosure. The navigation keys can be used by the user to access and hover over the menu of the ultrasound probe 102. The touch input display 204 displays the menu selections and also presents ultrasound images captured from the patient.

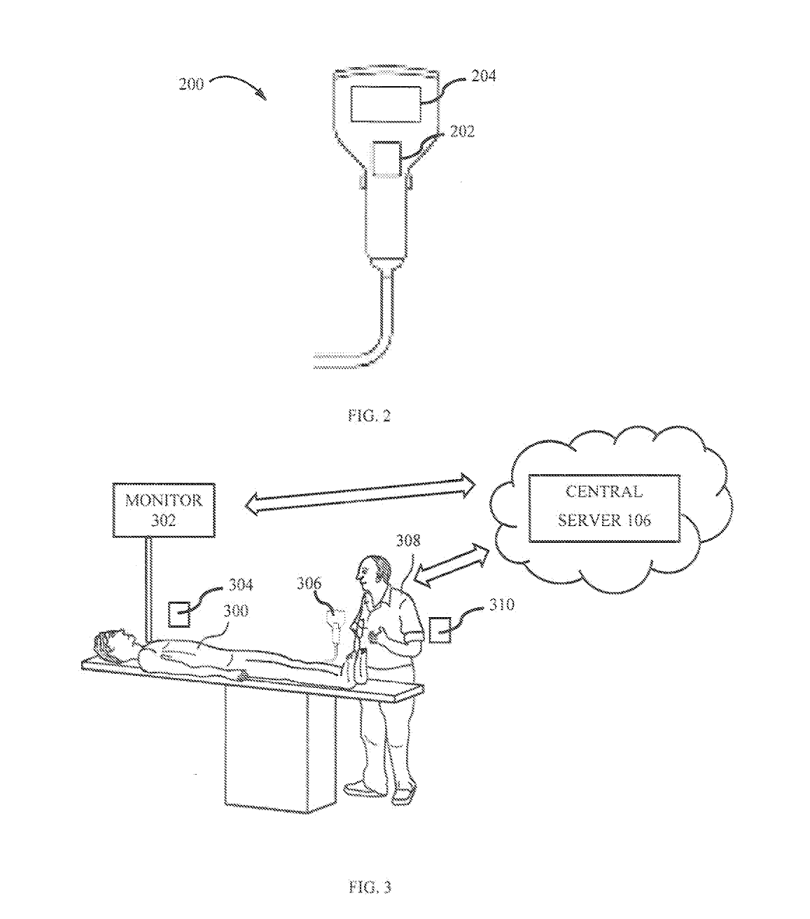

The ultrasound images captured from the patient may be also presented in a monitor 120 communicably connected to the central sever 106. FIG. 3 illustrates a patient 300 lying on a bed and a monitor 302 used for presenting ultrasound images captured from the patient according to an embodiment. The patient 300 or the bed may have an electronically readable card 304 that can retrieve the data associated with the patient 300. The electronically readable card 304 includes but not limited to patient data associated with the patient 300 and an IP address associated with the monitor 120. The electronically readable card 304 (hereinafter referred to as ‘card 304’) is detected by an ultrasound probe 306 that commences communication with the card 304. The card 304 is detected when it is at a NFC range of the ultrasound probe 306. The patient data and IP address of the monitor 120 retrieved or received from the card 304 at the ultrasound probe 306 is transmitted to a central server 106. The central server 106 sends set-up information for configuring the ultrasound probe 306. The set-up information enables the ultrasound probe 306 to be configured so to perform suitable ultrasound imaging on the patient. The imaging presets for ultrasound imaging may vary from person to person due to body structure and/or health complications and health checkup needs associated with the patient. For example if the person has high body mass then signal strength and specification of the ultrasound probe used may vary.

In another embodiment the card 304 may include patient ID, this patient ID may be communicated by the ultrasound probe 306 to the central server 106. The patient ID may be a unique ID provided to the patient upon registering with a hospital. Based on the patient ID the central server 106 retrieves the patient data and also the corresponding set-up information. The set-up information is then communicated to the ultrasound probe 306. In this embodiment there is no transfer of the patient data from the ultrasound probe 306 to the central server 106 and moreover the patient data need not be stored in the card 304 thereby making the need of less capacity electronically readable card for the patient.

A person 308 (such as a doctor or a technician) may also require authentication for using the ultrasound probe 306. The person 308 may have an electronically readable card 310 (also referred to as ‘card 310’) that may be read by the ultrasound probe 306 to retrieve authentication data of the person 308. Based on the authentication data the person 308 is authenticated and can start using the ultrasound probe 306. The authentication data may be retrieved from the card 310 using one of a NFC technique, a RFID technique, a QR reading technique and a barcode reading technique.

Now referring back to the ultrasound image captured, in an embodiment the ultrasound images may be processed by the ultrasound probe 306 (i.e. more specifically ultrasound signals received from the patient's body are processed to form the ultrasound images) and transmitted to the monitor 302. The monitor 302 presents the ultrasound images to the person 308 for examining the condition of the patient 300. The ultrasound probe 306 communicates with the monitor 302 over a wireless network. The wireless network may be a Wi-Fi network. In an embodiment the ultrasound signals are processed in the ultrasound probe 306 and the processed ultrasound signals may be send to the central server 106 to be converted into ultrasound images. The ultrasound images are then communicated to the monitor 302. The monitor 302 communicates over the wireless network with the central server 106. The wireless network may be but not limited to, a Wi-Fi network, a wireless LAN network, a 3G network and a 4G network. Further even though a single central server is shown in FIGS. 1 and 3 this is merely an exemplary embodiment and thus it may be envisioned that there can be multiple servers located in different regions and can communicate with the ultrasound probe 306. These servers may be for example standalone servers and cloud servers.

Once the person 308 completes examination of the patient 300 then the person 308 i.e. doctor can move to another patient and perform the same procedure using an electronically readable card of this patient. The set-up information for this patient may be completely different from the patient 300. For instance an ultrasound procedure performed on the patient 300 may be obstetric imaging and this patient may need another ultrasound procedure i.e. cardiac imaging. Accordingly once the new patient is attended the set-up information for the cardiac imaging is send to the ultrasound probe 306 to configure for performing the procedure.

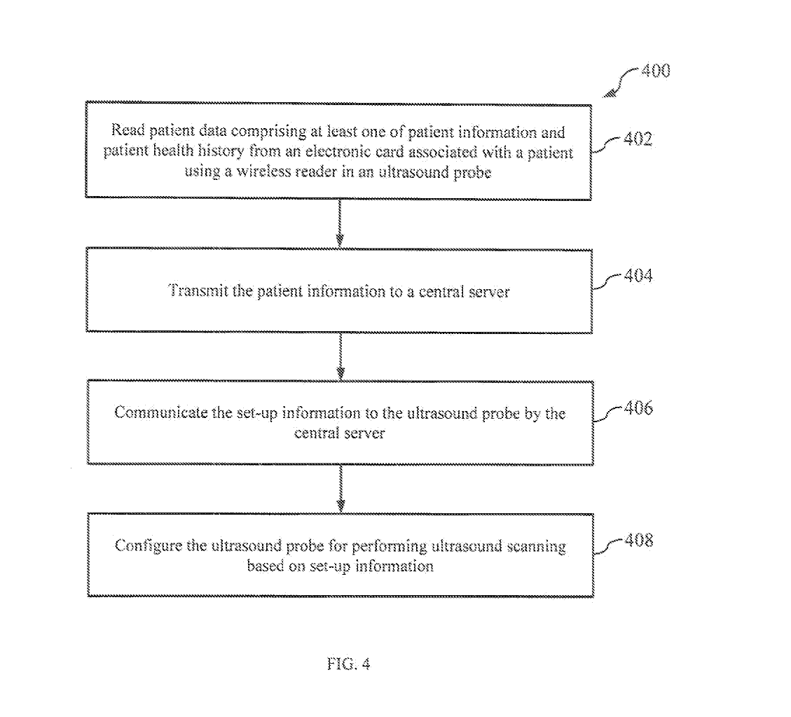

FIG. 4 illustrates a method 400 for managing automated patient data entry in an ultrasound device according to an embodiment. The method 400 includes reading patient data from an electronically readable card associated with a patient using a wireless reader in an ultrasound probe at block 402. The wireless reader may be activated and deactivated based on user input. The patient data may include but not limited to patient health history and patient information. The patient data is then transmitted to a central server at block 404. The central server communicates set-up information to the ultrasound probe based on the patient data received at block 406. The ultrasound probe is configured for performing ultrasound scanning on the patient based on the set-up information at block 408. The ultrasound probe is used by a user (also referred to as ‘person’) who needs to be authenticated in the ultrasound probe. The ultrasound probe communicates with the central server through a wireless communicator in the ultrasound probe. This is described in detail in conjunction with FIG. 1 and FIG. 3.

From the foregoing, it will appreciate that the above disclosed a system for automated management of data entry for a patient in an ultrasound device provide numerous benefits to healthcare enterprises, such as improved way of registering the patient data in a hospital environment even when patient is taken for scanning and examination. As the patient data is collected and registered at the time of performing an examination on the patient any delays for examining a patient during any emergency can be handled. Moreover manual entry of patient data can be reduced. Further the process of patient data entry is also more secure as the confidentiality is maintained.

This written description uses examples to disclose the invention, including the best mode, and also to enable any person skilled in the art to practice the invention, including making and using any computing system or systems and performing any incorporated methods. The patentable scope of an embodiment of the present invention defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An ultrasound probe for use with a patient, comprising:
a wireless reader configured to read a patient data comprising at least one of patient information and patient health history from an electronically readable card associated with the patient; and
a controller configured to:
transmit the patient data to a central server,
receive set-up information from the central server, and
set-up the ultrasound probe based on the set-up information.

2. The ultrasound probe of claim 1, wherein the wireless reader is one of a near field communication wireless reader, an RFID reader, a QR code reader, and a barcode reader.

3. The ultrasound probe of claim 1, wherein the set-up information comprises scan presets associated with the patient for performing ultrasound scanning on the patient.

4. The ultrasound probe of claim 1, wherein the controller comprises an authenticator configured to authenticate a user over a near field communication network for using the ultrasound probe.

5. The ultrasound probe of claim 1, further comprising an activation unit configured to activate and deactivate the wireless reader based on user input.

6. The ultrasound probe of claim 5, further comprising an activation button configured to enter the user input.

7. The ultrasound probe of claim 1, further comprising a wireless communicator communicating with the controller, wherein the wireless communicator facilitates communication between the controller and the central server.

8. The ultrasound probe of claim 1, further comprising an indicator configured to indicate at least one of patient data being successfully read from the electronically readable card, patient data being sent to the central server, and patient data being received from the central server.

9. The ultrasound probe of claim 8, wherein the indicator is at least one of an audible sound and a display.

10. The ultrasound probe of claim 9, wherein the audible sound is a beep and the display is an LED.

11. The ultrasound probe of claim 1, further comprising at least one of navigation keys and a touch input display.

12. A system for automated management of data entry for a patient in an ultrasound device, the system comprising:
an ultrasound probe comprising:
a wireless reader configured to read patient data comprising at least one of patient information and patient health history from an electronically readable card associated with the patient; and
a controller configured to:
configure the ultrasound probe for performing ultrasound scanning based on set-up information, and
transmit the patient data to a remote location; and
a central server at the remote location, the central server configured to:
receive the patient data from the controller, and
communicate the set-up information to the controller based on the patient data.

13. The system of claim 12, further comprising a monitor configured by the central server based on the patient information, wherein the monitor is positioned proximal to a patient bed and presents images captured using the ultrasound probe.

14. The system of claim 12, wherein the set-up information comprises scan presets associated with the patient for performing the ultrasound scanning on the patient.

15. The system of claim 12, wherein the controller comprises an authenticator configured to authenticate a user over a near field communication network for using the ultrasound probe, wherein the wireless reader is further configured to collect authenticating data from an electronically readable card associated with the user, wherein the authenticating data is used for user authentication.

16. The system of claim 15, further comprising an indicator configured to indicate at least one of patient data being successfully read from the electronically readable card associated with the patient, patient data being sent to the central server, and patient data being received from the central server, wherein the indicator is at least one of an audible sound and a display, wherein the audible sound is a beep and the display is an LED.

17. The system of claim 12, further comprising an activation unit configured to activate and deactivate the wireless reader based on user input.

18. The system of claim 17, further comprising:
an activation button configured to activate and deactivate the wireless reader by a user; and
a wireless communicator communicating with the controller, wherein the wireless communicator facilitates communication between the controller and the central server.

19. A method of managing automated patient data entry in an ultrasound device, the method comprising:
reading patient data comprising at least one of patient information and patient health history from an electronic card associated with a patient using a wireless reader in an ultrasound probe;
transmitting the patient information to a central server;
communicating set-up information to the ultrasound probe by the central server; and
configuring the ultrasound probe for performing ultrasound scanning based on the set-up information.

20. The method of claim 19, further comprising:
authenticating a user over a near field communication network for using the ultrasound probe;
activating and deactivating the wireless reader based on user input; and
facilitating a communication between a controller of the ultrasound probe and the central server through a wireless communicator.
